(19) United States
(12) Patent Application Publication
(10) Pub. No.: US 2026/0053637 A1
(43) Pub. Date: Feb. 26, 2026

(54) EXPANDABLE INTERVERTEBRAL CAGE WITH MULTI-CYCLING FLEXURES

(71) Applicant: Octagon Spine LLC, Bellevue, WA (US)

(72) Inventors: Omar F. Jimenez, Seattle, WA (US); Yefim I. Safris, Golden Valley, MN (US)

(21) Appl. No.: 18/813,280

(22) Filed: Aug. 23, 2024

(51) Int. Cl. A61F 2/44; A61F 2/30

(52) U.S. Cl. CPC A61F 2/447; A61F 2002/30326; A61F 2002/30433

(57) ABSTRACT

Disclosed herein are systems and methods for intervertebral body fusion that provide more robust support within the disc space. Intervertebral body fusion devices can have a unitary monolithic body including a plurality of body segments interconnected with each other by flexure members. Devices may be configured to be inserted through an opening in a compressed configuration and then expanded within the disc space to an expanded configuration. In the expanded configuration, devices can have a greater mediolateral or transverse footprint with regard to the disc space. This wider footprint provides greater support for the vertebrae relative to the size of the opening through which the device is inserted. In the event the device must be expanded or compressed multiple times during the insertion process, the flexures of the device are resistant to failure or fracture.

### TECHNICAL FIELD

The present disclosure relates to the fusion of vertebral bodies. More specifically, the present disclosure relates to devices and associated methods for the fusion of vertebral bodies that provide robust spinal support in a less invasive manner.

### BACKGROUND

The concept of intervertebral fusion for the cervical and lumbar spine following a discectomy was generally introduced in the 1960s. It involved coring out a bone graft from the hip and implanting the graft into the disc space. The disc space was prepared by coring out the space to match the implant. The advantages of this concept were that it provided a large surface area of bone-to-bone contact and placed the graft under loading forces that allowed osteoconduction and induction enhancing bone fusion. However, the technique is seldom practiced today due to numerous disadvantages including lengthy operation time, destruction of a large portion of the disc space, elevated risk of nerve injury, and hip pain after harvesting the bone graft.

Presently, at least two devices are commonly used to perform the intervertebral portion of an intervertebral body fusion: the first is the distraction device and the second is the intervertebral body fusion device, often referred to as a cage. Cages can be implanted as standalone devices or as part of a circumferential fusion approach with pedicle screws and rods. The concept is to introduce a distraction device that will distract a collapsed disc in a generally axial direction, decompress the nerve root, and allow load sharing to enhance bone formation, and then implant an intervertebral fusion device that is small enough to allow implantation with minimal retraction and pulling on nerves.

In a typical intervertebral body fusion procedure, a portion of the intervertebral disc is first removed from between the vertebral bodies. This can be done through either a direct open approach or a minimally invasive approach. Disc shavers, pituitary rongeours, curettes, and/or disc scrapers can be used to remove the nucleus and a portion of either the anterior or posterior annulus to allow implantation and access to the inner disc space. The distraction device is inserted into the cleared space to enlarge the disc space such that the vertebral bodies are separated in a generally axial direction by actuating the distraction device. Enlarging the disc space is important because it also opens the foramen where the nerve root exists. It is important that during the distraction process one does not over-distract the facet joints. An intervertebral fusion device is next inserted into the distracted space and bone growth factor, such as autograft, a collagen sponge with bone morphogenetic protein, or other bone enhancing substance may be inserted into the space within the intervertebral fusion device to promote the fusion of the vertebral bodies.

Intervertebral distraction and fusion can be performed through anterior, posterior, oblique, and lateral approaches. Each approach has its own anatomical challenges, but the general concept is to fuse adjacent vertebra in the cervical thoracic or lumbar spine. Devices have been made from various materials. Such materials include cadaveric cancellous bone, carbon fiber, titanium and polyetheretherketone (PEEK). Devices have also been made into different shapes such as a bean shape, football shape, banana shape, wedge shape and a threaded cylindrical cage.

As with all minimally invasive surgeries, a primary goal is to provide equivalent or near equivalent treatment as more invasive surgical techniques but with less discomfort, recovery time, etc., for the patient. One problem with minimally invasive intervertebral fusion procedures is that the limited size of the surgical access limits the size of the implant(s) that can be inserted. While devices that are vertically expandable in a generally axial direction have addressed some of these issues by being able to be inserted through a smaller opening and then made taller in a generally axial direction within the disc space, such devices are still limited in the transverse footprint that can be covered within the disc space which can affect the stability of the device within the disc space and limits the area for bone grown.

### SUMMARY

Non-limiting examples of the present disclosure provide an expandable intervertebral body fusion device. The expandable intervertebral body fusion device can include a unitary monolithic body having a plurality of body segments coupled to each other with flexure members and an opening defined between the plurality of body segments, wherein the body is configured to automatically mediolaterally expand from a compressed configuration to an expanded configuration, causing the plurality of body segments to generally move away from each other and expand the opening between the plurality of body segments such that the body forms a greater mediolateral footprint in the expanded configuration than in the compressed configuration.

In embodiments, the plurality of body segments includes adjacent projections and grooves that form tongue and groove connections between adjacent mediolateral body segments when the body is in the expanded configuration, the tongue and groove connections providing increased resistance of the body to shear and torsional forces.

One example of the present disclosure provides an expandable intervertebral body fusion device, comprising a unitary monolithic body having a plurality of body segments coupled to each other with flexure members and an opening defined between the plurality of body segments. In some embodiments, the expandable intervertebral body fusion device includes a first core extending from a first end body into the opening and a second core extending from a second end body segment into the opening. The body is configured to be mediolaterally expanded from a compressed configuration to an expanded configuration causing one or more mediolateral body segments on the lateral side and the one or more mediolateral body segments on the medial side to generally move away from each other and expand the opening between the plurality of body segments such that the body forms a greater mediolateral footprint in the expanded configuration than in the compressed configuration, and wherein the first and second cores are brought together within the opening and further expansion of the body is prevented by the interaction between the first and second cores.

One example of the present disclosure provides a method for operating an expandable intervertebral body fusion device providing a unitary monolithic fusion device in a collapsed configuration implanted into a body of a patient in the collapsed configuration, wherein the device expands into an expanded configuration in the body of the patient in which the device is implanted. In some instances, it may be necessary for the unitary monolithic fusion device to be collapsed and expanded multiple times during operation to ensure that the unitary monolithic fusion device is correctly placed within the body of the patient. In such instances the unitary monolithic fusion device described herein is configured to be expanded and collapsed multiple times without breaks or fractures appearing in the flexure members.

The above summary is not intended to describe each illustrated embodiment or every implementation of the subject matter hereof. The figures and the detailed description that follow more particularly exemplify various embodiments.

### BRIEF DESCRIPTION OF DRAWINGS

The subject matter disclosed herein may be more completely understood in consideration of the following detailed description of various embodiments in connection with the following figures.

FIG. 1 depicts an expandable intervertebral body fusion device in a collapsed configuration in accordance with the current disclosure.

FIG. 2 depicts the expandable intervertebral body fusion device of FIG. 1 in an expanded configuration.

FIG. 3 depicts an alternate embodiment of an expandable intervertebral body fusion device in a collapsed configuration in accordance with the current disclosure.

FIGS. 4-5 depict an expandable intervertebral body fusion device having a greater lateral height in accordance with the current disclosure.

FIGS. 6-7 depict a convex flexure in accordance with the current disclosure.

FIG. 8 depicts a concave flexure in accordance with the current disclosure.

FIG. 9 is a cutaway view of the embodiment of FIG. 1 illustrating a stabilizing screw.

FIG. 10 is a cross sectional view depicting various surgical approaches for inserting an expandable intervertebral body fusion device in accordance with the current disclosure.

FIG. 11 depicts an intervertebral body fusion device in the collapsed configuration attached to an inserter in accordance with the current disclosure.

FIG. 12 depicts an intervertebral body fusion device in the expanded configuration attached to an inserter in accordance with the current disclosure.

FIG. 13 depicts an exploded view of the inserter of FIGS. 11-12.

While various embodiments are amenable to various modifications and alternative forms, specifics thereof have been shown by way of example in the drawings and will be described in detail hereafter. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Reference now will be made in detail to embodiments of the disclosure, one or more examples of which are illustrated in the drawings. Each example is provided by way of explanation in the disclosure and is not limited thereto. In fact, it will be apparent to those skilled in the art that various modifications and variations can be made in the present disclosure without departing from the scope of the disclosure. For instance, features illustrated or described as part of one embodiment can be used with another embodiment to yield a still further embodiment.

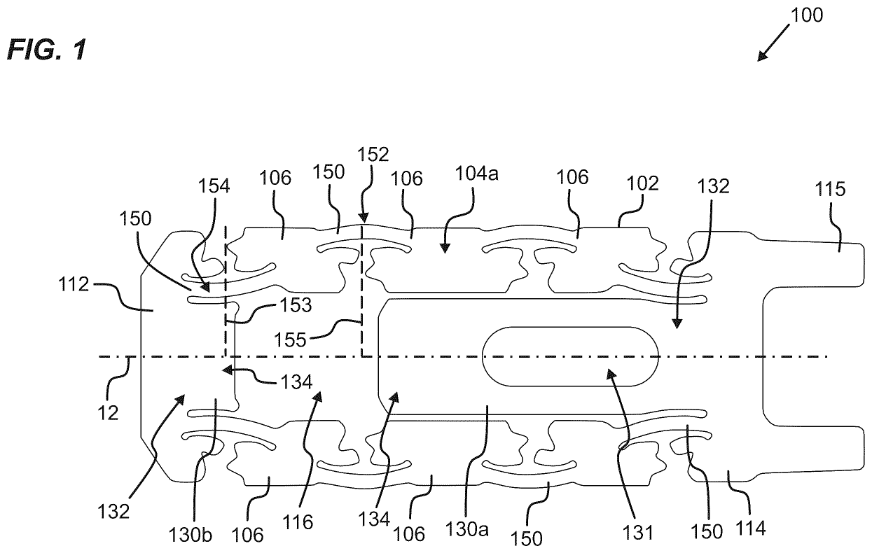

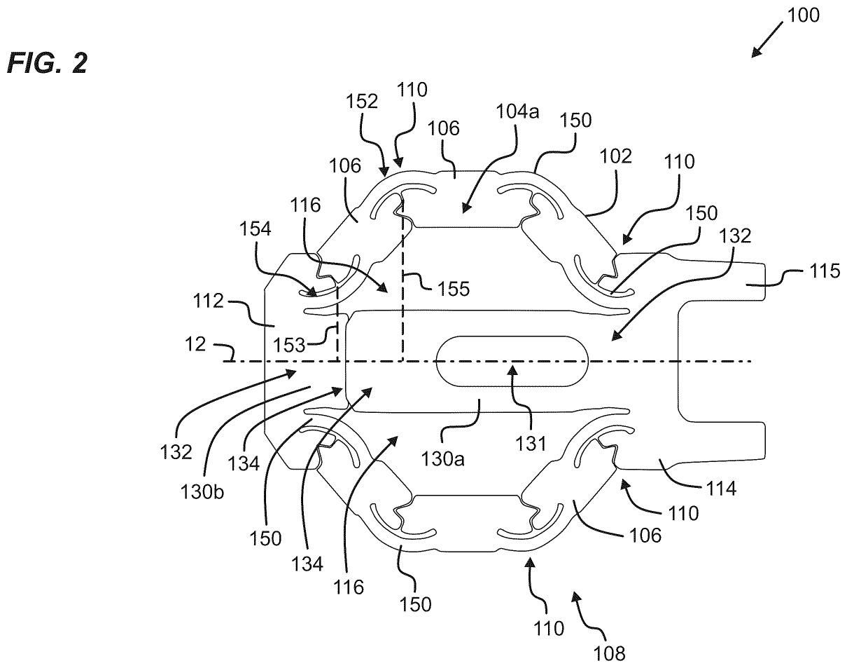

FIGS. 1 and 2 depict an expandable intervertebral body fusion device 100 according to an embodiment. FIG. 1 depicts device 100 in a collapsed configuration and FIG. 2 depicts device 100 in an expanded configuration. In practice, device 100 can be inserted into the disc space through a minimally invasive access in the collapsed configuration and then expanded inside of the disc space. In embodiments, device 100 can be inserted between adjacent vertebrae on its side such that when it is expanded in the disc space it expands horizontally/transversely to the disc space to enable device 100 to have a larger footprint within the disc space once device 100 is expanded. Alternatively, device 100 can be inserted between adjacent vertebrae vertically such that when it is expanded in the disc space it expands vertically/longitudinally within the disc space to enable device 100 to expand the disc space once device 100 is expanded. Device 100 is therefore able to occupy more lateral to medial and anterior to posterior space within the disc space relative to the size of the access.

In one embodiment, in its insertion and un-expanded state, device 100 is approximately 8 mm in height, approximately 11 mm in width (e.g., one end may have a larger width than the other) and approximately 31 mm in length. Device 100 can have many heights from 6 mm up to 16 mm. In embodiments, the width can go from 8-29 mm and the length from 22 mm-42 mm. When device 100 is expanded, the height remains the same, but the width can double or nearly double (from 11.5 to 22 mm or 47%) and the length can go from 26 mm to 20 mm (16% decrease). Device 100 can have many lordotic angles from 0 to 15 degrees or higher; the most common being 0, 4, 6, or 12 degrees. The top and bottom of device 100 can have different shapes to better fit the endplates such as football shaped or domed. Also, the different segments of device 100, separated by flexures 108, can be tailored or cut by wire EDM, 3D printed or machined, etc. to create different horizontal expanded states such as oval, elliptical, circular, bean shaped, banana shaped or many other polygons and non-polygon shapes. The mean disc height at the L3-4 level is 11.3 mm +/−1.8 mm, L4-5 11.3+/−2.1 mm and L5-S1 10.7+/−2.1 mm. The average circumference of the L4 endplate of a patent is about 141 mm and surface area 1,492 mm² above. Device 100 can have different footprints to try to fill the endplate or disc space circumference.

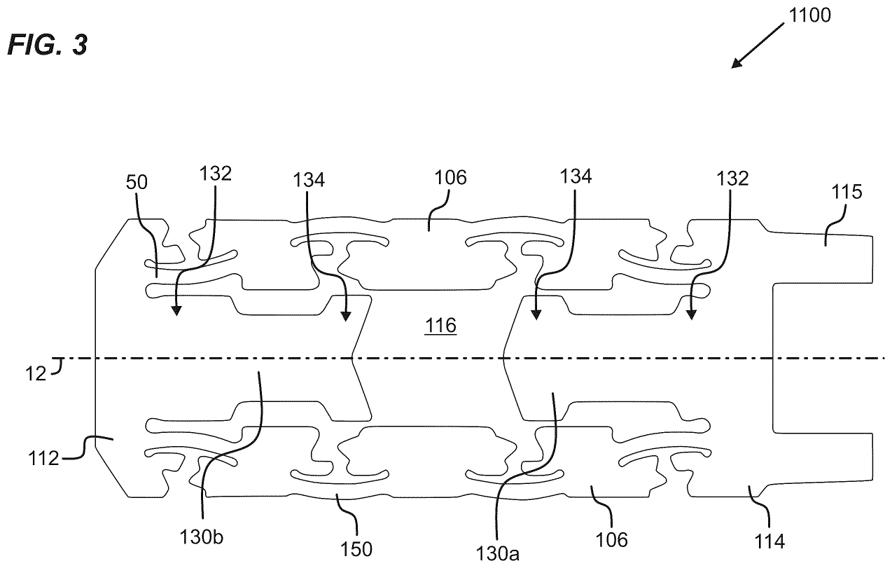

Referring now to FIG. 1, device 100 can include a device body 102. Generally, device body 102 is unitarily formed as a single monolithic construct, although multiple component embodiments are also considered to be within the scope of this disclosure. Device body 102 can include bearing surfaces 104, including an upper bearing surface 104a and a lower bearing surface 104b. As noted above, device 100 can be inserted generally on its side such that bearing surfaces 104a, 104b interface with and bear the load of the adjacent vertebrae. Device body 102 may further include a plurality of side body segments 106 unitarily connected to one another by flexures 150. Flexures 150 are discussed in greater detail with regard to FIGS. 3-5. Device body 102 may further include a first end body segment 112 and a second end body segment 114 that may also be connected to the side body segments 106 by flexures 150. In such embodiments, side body segments 106, end body segments 112, and flexures 150 define a continuous, unitary perimeter of device body 102. An interior space 116 is defined between the body segments 106, 112, 114. In some embodiments, device body 102 is configured such that, during insertion of device 100 by a surgeon, end body segment 112 is an anterior body segment which is distal to the surgeon, end body segment 114 is a posterior body segment which is proximal to the surgeon during insertion, and side body segments 106 are mediolateral body segments. Device 100 additionally includes central longitudinal axis 12 which extends between the first end body segment 112 and the second end body segment 114.

Referring now to FIG. 2, device 100 is shown in an expanded configuration. As device 100 is expanded, side body segments 106 on opposing sides of device body 102 are moved away from each other causing the device to expand medially and laterally, providing a larger area to facilitate bone grown within device 100. As device 100 is expanded, it forms a polygon 108 having vertices 110, wherein each of the vertices 110 is defined where each of the body segments 106, 112, and 114 connects to the adjacent body segment. Vertices 110 may be convex vertices or concave vertices, with the shape of the vertex being determined by configurations of the flexure 150 located at each of the vertices 110 with flexures 150 having a convex configuration 152 dictating convex vertices and flexures 150 having a concave configuration 154 dictating concave vertices. Device 100 further includes a first distance 153 from central longitudinal axis 12 to a center of a flexure 150 having a concave configuration 154 and a second distance 155 from central longitudinal axis 12 extending to a center of a flexure 150 having a convex configuration 152. In some embodiments, second distance 155 has a greater length than first distance 153 when device body 102 is in either the compressed configuration or the expanded configuration. In some embodiments, device 100 includes three side body segments 106 on each side such that device 100 includes a total of eight body segments such that polygon 108 is generally octagonal in shape when device 100 is in the expanded configuration as illustrated in FIG. 2. In other embodiments, device 100 may have greater or fewer side body segments on each side.

Device body 102 can further include one or more cores 130 located within interior space 116 including a first core 130a and a second core 130b. Each of the one or more cores 130 includes a base 132 and an interior end 134, wherein the base 132 is attached to an interior surface of one of first end body segment 112 or second end body segment 114 and interior end 134 extends inward from the base 132, terminating within interior space 116. In some embodiments one or more cores 130 includes a first core 130a and a second core 130b each having a particular axial length, wherein first core 130a extends from inwardly from the second end body segment 114 and second core 130b extends inwardly from the first end body segment 112. In some embodiments, further expansion of device 100 is prevented by the contact of one of interior ends 134 with an opposite interior surface of first end body segment 112 or second end body segment 114, or by contact of one of interior ends 134 with the other of interior ends 134. In some embodiments, cores 130 may be a locking bushing including locking projections configured to form a locking or restricting mechanism which prevents further expansion device 100. By preventing the overexpansion of device 100, cores 130 prevent damage which may be sustained by device body 102 as a result of over expansion. In some embodiments, the one or more cores 130 include one or more slots 131 through which a bone graft material may be infused. An alternative embodiment of the expandable intervertebral body fusion device 1100 is shown in FIG. 3, wherein device 1100 includes a first core 130a and a second core 130b. In some embodiments, additional or alternative locking mechanisms can be incorporated and interlock with a corresponding locking element, wherein alternative sizes, positions, and other mechanisms that can be sufficient to, in part, restrict the expansion of the structure providing alternative footprint sizes to fill the endplate or disc space circumference when implanted. Once device 100 is expanded, these locking mechanisms can prevent further expansion, which prevents damage to device body 102 that may otherwise result from over-expansion.

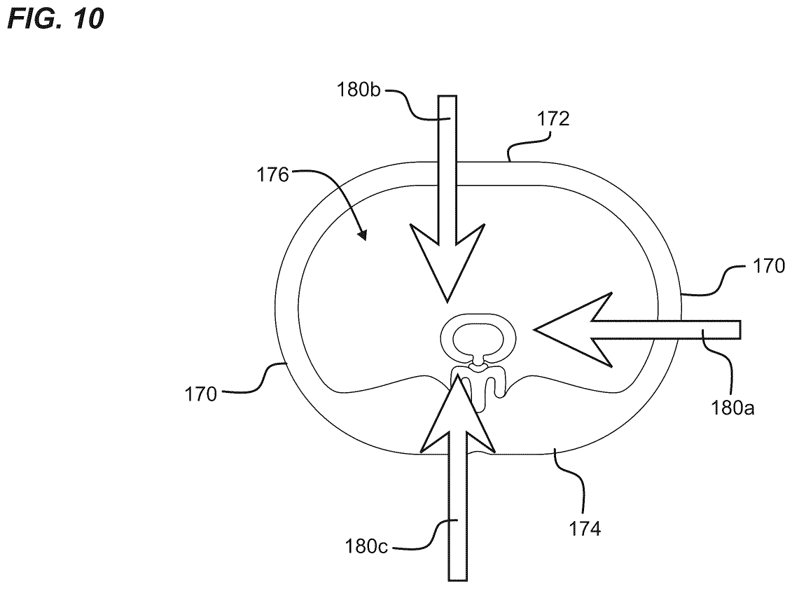

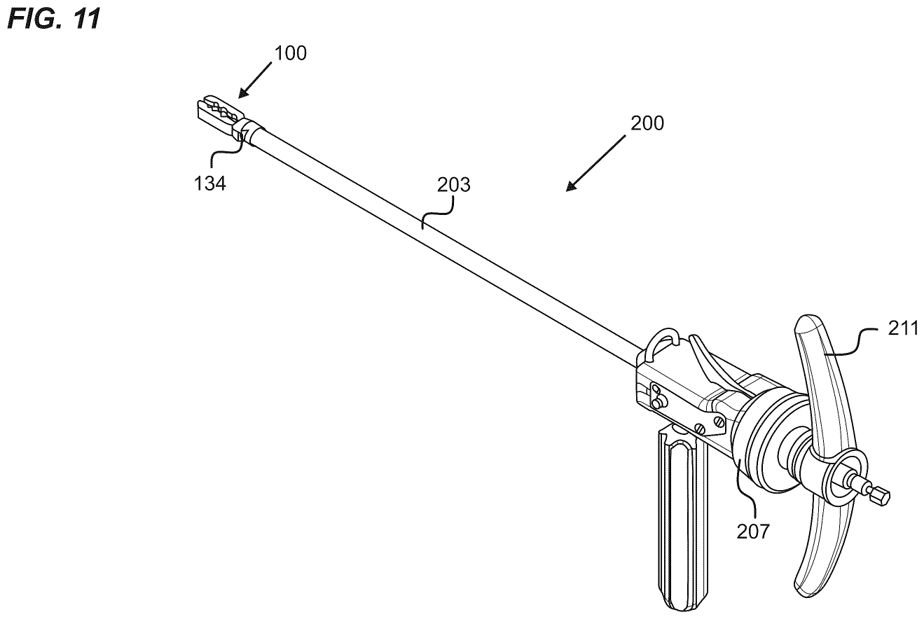

In some embodiments each of the flexures 150 have a greater height 164a than any other of the heights 164b of any other of the flexures 150. In some embodiments, the flexures 150 connecting each of the one or more mediolateral body segments 106 to any other of the one or more mediolateral body segments 160 on either the lateral side or the medial side of the device 100 have a greater height 164a than each of the other flexures such that the device 100 has a greater angle perpendicular to the plane of insertion in order to create angulation between adjacent vertebrae. In other embodiments, the flexures 150 connecting the first end body segment 112 to the one or more mediolateral body segments 106 have a greater height 164a than each of the other flexures 150 such that the device 100 creates an angle along the axial plane of insertion. In still other embodiments, the flexures 150 connecting the second end body segment 114 to the one or more mediolateral body segments 106 have a greater height 164a than each of the other flexures 150 such that the device 100 creates an angle along the axial plane of insertion. The angulation of the device 100 may be selected depending on the needs of the patient and the desired outcome. Similarly, in some embodiments, the exact placement of device 100 may vary depending on the desired outcome. Placement of device 100 is described in greater detail with regards to FIG. 10.

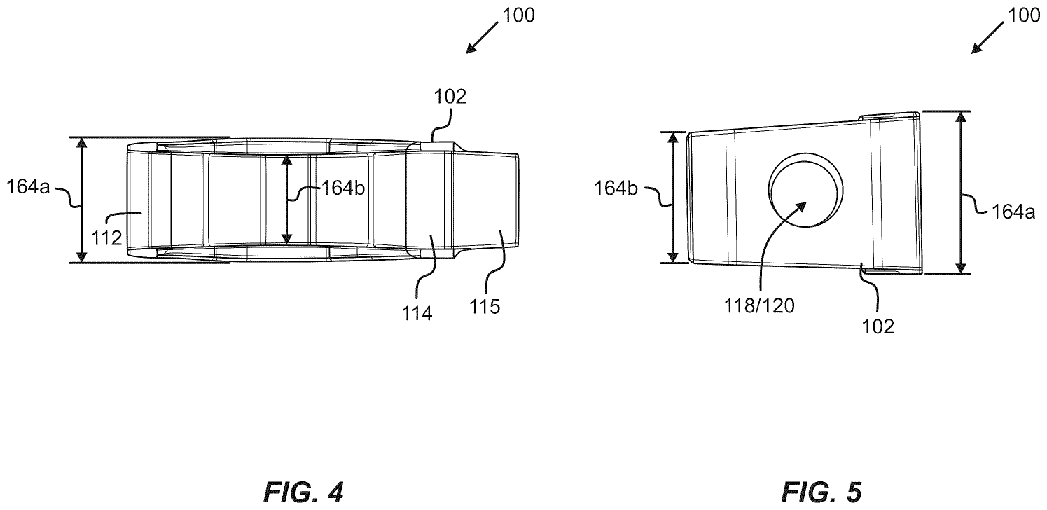

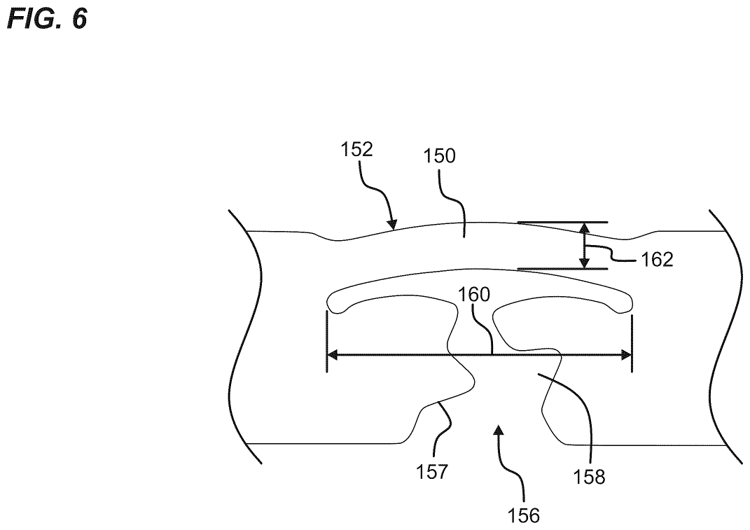

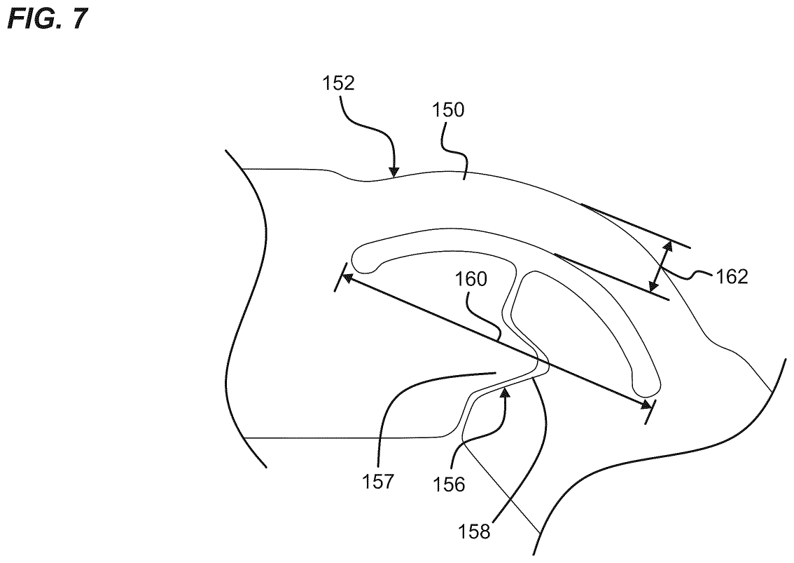

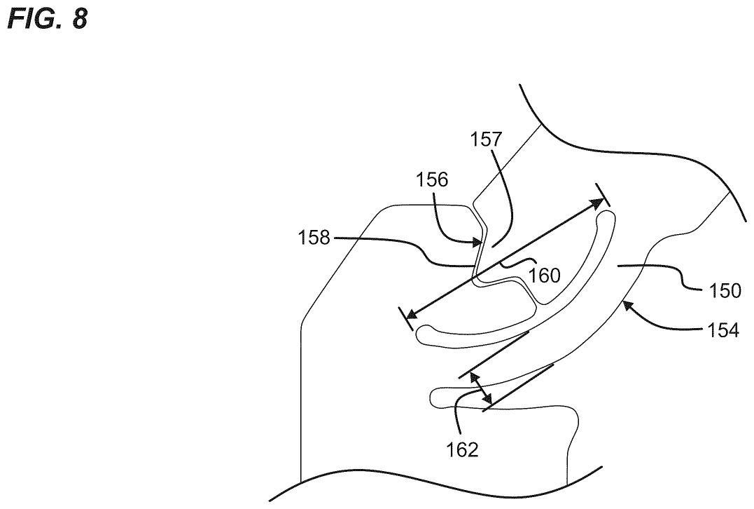

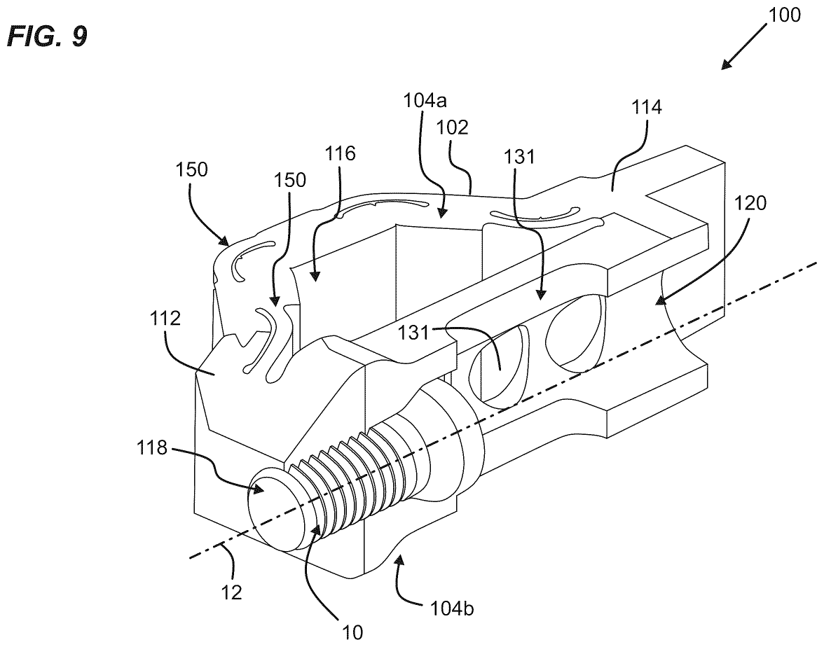

FIGS. 6-8 illustrate flexures 150 in greater detail. In some embodiments flexures 150 are configured in either a convex configuration 152 or in a concave configuration 154, wherein the convex configuration 152 extends outward toward an outer edge of device body 102 and the concave configuration 154 is recessed inward from an outer edge of the device body 102. Flexures 150 are further configured to remain in either the convex configuration 152 or the concave configuration 154 when device 100 is in both the compressed configuration and the expanded configuration. FIGS. 3 and 4 illustrate the convex configuration 152 in both compressed and expanded configurations. In some embodiments, the flexures 150 connecting the first end body segment 112 and the second end body segment 114 to the one or more mediolateral body segments 106 are configured in the concave configuration 152, and the flexures 150 connecting each of mediolateral body segments 106 to any other one of the one or more mediolateral body segments 106 are configured in the convex configuration 154. In some embodiments each of the flexures 150 have a length 160, a thickness 162, wherein the ratio of thickness 162 to length 160 is less than 0.2. In embodiments, flexures may include adjacent projections 157 and grooves 158 that form a tongue and groove system 156 between adjacent body segments 106, 112, and 114 when the body is in the expanded configuration, wherein the tongue and groove system 156 provides increased resistance of the body to shear and torsional forces.

Referring now to FIG. 9, device 100 is illustrated in the expanded configuration. In some embodiments each of first end body segment 112 and second end body segment 114 may include an opening that assists in insertion and/or expansion of device. In one embodiment, first end body segment 112 includes a first opening 118 and second end body segment 114 includes a second opening 120. A stabilizing element, such as a screw 10 can be extended through second opening 120 and one or more cores 130 and into the first opening 118, which, in the case of the stabilizing element being a screw, may be threaded to interface with the threads of the screw. Second end body segment 114 can further include a pair of flanges 115 in which a screw head of a stabilizing screw 10 can be contained. Flanges 115 can also define outer gripping surfaces that can be engaged by an insertion element used to insert device 100 into the patient's body. In some embodiments, first end body segment 112 can be tapered to facilitate insertion of device 100 into the disc space through the minimally invasive access opening.

In some embodiments, a small incision is made in the patient's body such that the device 100 may be appropriately inserted. The incision may be made in a lateral side 170 of the patient's body 176, from a posterior side 174 of the patient's body 176, or an anterior side 172 of the patient's body 176. The device 100 may then be inserted through the patient's body 176 and into an appropriate position along an axis of insertion 180. In some embodiments, the device 100 is inserted between an upper vertebral disc and a lower vertebral disc while in the compressed configuration and subsequently opened into the expanded configuration.

In some instances, it may be necessary for the device 100 to be relocated from its initial placement between the vertebral discs during the insertion process. In such instances, the device 100 may be returned to the compressed configuration, the position of device 100 adjusted, and the device 100 opened into the expanded configuration. In some instances, it may be necessary to repeat this process multiple times during the insertion process. In some embodiments flexures 150 having the configuration described herein are multi-cycling flexures which may transition between the compressed configuration and the expanded configuration multiple times without fracturing or weakening. Such embodiments are advantageous for reducing potential complications which may arise during or subsequent to the insertion of device 100; for example, as a result of a broken device 100 or debris generated therefrom.

In some embodiments, device 100 may be implanted within the patient's cervical spine between a first cervical vertebrae and a second cervical vertebrae. In other embodiments, device 100 may be implanted within other regions of patient's spine. FIG. 10 illustrates a variety of approaches which may be used when inserting device 100.

As way of example, in a patient requiring Anterior Lumbar Interbody Fusion (ALIF), device 100 may be inserted through the anterior 172 of the patient's body 176 and placed between the target vertebral discs along an axis of insertion 180b such that the first end body segment 112 is located closest to the patient's posterior 174 and the second end body segment 114 is located closest to the incision in the patients anterior. In this instance, the second end body segment 114 may have a greater height 164a than first end body segment 112 or any of the side body segments 106 in order to create an appropriate lordotic angle. In another example, in a patient requiring Lateral Lumbar Interbody Fusion (LLIF) device 100 may be inserted through a side approach, wherein the incision is made in a lateral side 170 of the patient's body 176 and device 100 is placed between the target vertebral discs along an axis of insertion 180a such that the second end body segment 114 is located closest to the incision. In this approach, a first set of side body segments 106a are located more proximal to and generally parallel with the patient's anterior side 172 and a second set of side body segments 106b opposite to the first set of side body segment 106a are more proximal to and generally parallel with the patient's posterior side 174. The first set of body segments 106a in this example have a greater height 164a, as seen in FIGS. 4-5, such that an appropriate lordotic angle is created. In still another example, in a patient requiring Posterior Lumbar Interbody Fusion (PLIF), device 100 may be inserted through the posterior 174 of the patient's body 176 and placed between the target vertebral discs along an axis of insertion 180c such that the first end body segment 112 is located closest to the patient's anterior 172 and the second end body segment 114 is located closest to the incision in the patient's posterior 174. In this instance, the first end body segment 112 may have a greater height 164a than the second end body segment 114 or any of the side body segments 106 in order to create an appropriate lordotic angle. Additional surgical approaches are possible; likewise, device 100 may have a variety of orientations beyond those described in the examples herein.

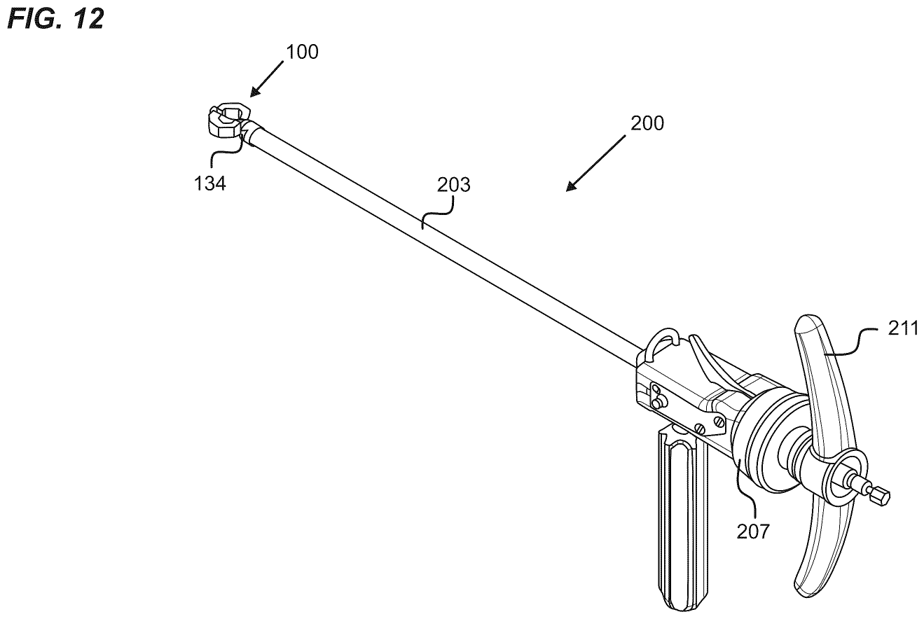

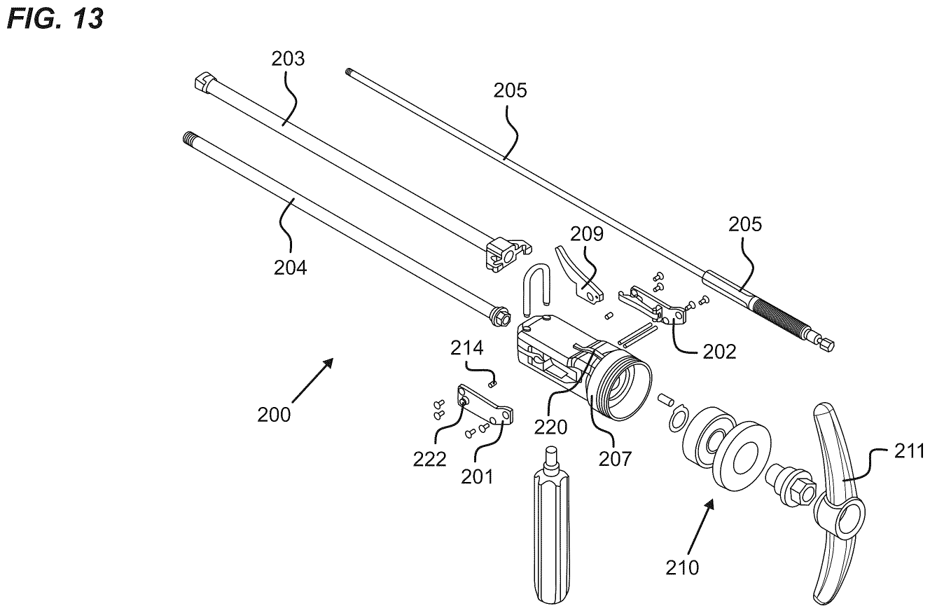

FIGS. 11-13 depict an embodiment of an insertion device 200 that can be used to implant and extract an expandable intervertebral body fusion device as disclosed herein. In embodiments, the insertion device 200 can be used to assist in the expansion of the body 102. In embodiments, the insertion device 200 can also assist in undoing the expanded implant to its original insertion shape for removal. Insertion device 200 can include an attachment rod 203 configured to grip the outer surfaces of flanges 115 of device 100, a stabilizing rod 204 having a threaded distal end configured to attach to the threaded inner surfaces of flanges 115 and to be extended through attachment rod 203 and an expansion rod 205 configured to be extended through stabilizing rod 204 and having a threaded distal end configured to interface with first opening 118 in device 100 to expand device 100. In operation, expansion rod 205 can be actuated, at least in part, by rotating handle 211 that interfaces with expansion rod 205 and housing 207 via handle assembly 210. To prevent rotation of expansion rod 205, key lock 209 can be actuated to insert key lock 209 into a slot 220 in expansion rod 205. Rotation of handle 211 can, therefore, help cause the expansion element 205 to pull the distal end of the implant back towards the proximal end, which can expand the implant. The expanded implant can also be, at least in part, reversed into its insertions state (straightened) by rotating handle 211 counterclockwise. This would assist in the removal of an expanded implant after insertion into the disc space. Following expansion, one or more buttons 222 can be actuated to disengage spring 214 loaded latches 201, 202 from housing 207, to enable housing 207 to be detached. Expansion rod 205 can also be detached by releasing the key lock and rotating the rod to disengage from the threaded distal opening of the implant. Next, the stabilizing rod 204 can be detached. One or more of bone graft and a stabilizing screw 10 (as described above) can be inserted through attachment rod 203 before the attachment rod 203 is detached to complete the insertion and expansion procedure.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations, and locations, etc., have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An expandable intervertebral body fusion device, comprising:
a unitary monolithic body having a plurality of body segments connected to each other with flexure members and an interior space defined between the plurality of body segments, including:
an anterior body segment;
a posterior body segment;
one or more mediolateral body segments extending between the anterior body segment and the posterior body segment along both a lateral side and a medial side of the anterior body segment;
an opening formed in each of the anterior body segment and the posterior body segment;
wherein the body is configured to be mediolaterally expanded from a compressed configuration to an expanded configuration by interaction of an expansion tool with at least one of the openings in the anterior body segment and posterior body segment causing the one or more mediolateral body segments on the lateral side and the one or more body segments on the medial side to generally more away from each other and expand the interior space between the plurality of body segments such that the body has greater mediolateral footprint in the expanded configuration than in the compressed configuration
wherein each of the flexures are configured in either a concave configuration or in a convex configuration, and wherein each of the flexures remain in either the concave configuration or the convex configuration in both the compressed configuration and the expanded configuration.

2. The expandable intervertebral body fusion device of claim 1, wherein the opening formed in each of the anterior body segment and the posterior body segment is a threaded opening.

3. The expandable intervertebral body fusion device of claim 1 further comprising a core extending from one of the anterior body segment and the posterior body segment into the interior space in the body, wherein further expansion of the body is prevented by interaction of the core with the other of the anterior body segment and the posterior body segment.

4. The expandable intervertebral body fusion device of claim 3 wherein the core comprises a first core and a second core having different axial lengths, wherein each different axial length is configured to permit a predetermined amount of expansion of the body.

5. The expandable intervertebral body fusion device of claim 3 wherein the core includes one or more slots through which a bone graft material may be infused.

6. The expandable intervertebral body fusion device of claim 3, further including a stabilizing screw inserted through the core along an axis extending from a proximal end of the core to a distal end of the core.

7. The expandable intervertebral body fusion device of claim 1, wherein the flexures have a height, a thickness, and a length, and wherein the ratio of thickness to length is less than 0.2.

8. The expandable intervertebral body fusion device of claim 6, wherein the flexures connecting the anterior body segment to the one or more mediolateral body segments have a greater height than each of the other flexures.

9. The expandable intervertebral body fusion device of claim 6, wherein the flexures connecting the posterior body segment to the one or more mediolateral body segments have a greater height than each of the other flexures.

10. The expandable intervertebral body fusion device of claim 6, wherein the flexures connecting each of the one or more mediolateral body segments to any other of the one or more mediolateral body segments on either the lateral side or the medial side of the anterior body segment have a greater height than each of the other flexures.

11. The expandable intervertebral body fusion device of claim 1, wherein the flexures connecting the anterior body segment and the posterior body segment to the one or more mediolateral body segments are configured in the concave configuration, and wherein the flexures connecting each of the one or more mediolateral body segments to any other one of the one or more mediolateral body segments are configured in the convex configuration.

12. The expandable intervertebral body fusion device of claim 1, wherein a distance from a central axis extending between the anterior body segment and the posterior body segment to a center of a flexure configured in a convex configuration is greater than the distance from the central axis to a center of a flexure configured in a concave configuration when the body is in the compressed configuration.

13. The expandable intervertebral body fusion device of claim 1, wherein each of the plurality of body segments form a tongue and groove structure with each adjacent one of the plurality of body segments at each of the vertices of the polygon.

14. An expandable intervertebral body fusion device, comprising:
a unitary monolithic body having a plurality of body segments coupled to each other with flexure members and an interior space defined between the plurality of body segments;
wherein the body is configured to be mediolaterally expanded from a compressed configuration to an expanded configuration causing the plurality of body segments to generally move away from each other to expand the interior space between the plurality of body segments such that the body forms a greater mediolateral footprint in the expanded configuration than in the compressed configuration;
wherein the flexures configured in either a concave configuration or in a convex configuration, and wherein the each of the flexures remain in either the concave configuration or the convex configuration in both the compressed configuration and the expanded configuration.

15. The expandable intervertebral body fusion device of claim 14, further comprising at least one core extending from one or more of the anterior body segment and the posterior body segment into the opening in the body.

16. The expandable intervertebral body fusion device of claim 15 wherein the at least one core comprises a first core and a second core having different axial lengths, wherein each different axial length is configured to permit a predetermined amount of expansion of the body.

17. The expandable intervertebral body fusion device of claim 15 wherein the core includes one or more slots through which a bone graft material may be infused.

18. The expandable intervertebral body fusion device of claim 14, wherein the flexures have a height, a thickness, and a length, and wherein the ratio of thickness to length is less than 0.2.

19. The expandable intervertebral body fusion device of claim 14, wherein a distance from a central axis extending between the anterior body segment and the posterior body segment to a center of a flexure configured in a convex configuration is greater than the distance from the central axis to a center of a flexure configured in a concave configuration when the body is in the compressed configuration.

20. The expandable intervertebral body fusion device of claim 14, wherein each of the plurality of body segments form a tongue and groove structure with each adjacent one of the plurality of body segments at each of the vertices of the polygon.
